(19) United States
(12) Patent Application Publication
(10) Pub. No.: US 2025/0351889 A1
(43) Pub. Date: Nov. 20, 2025

(54) UNDERWEAR

(71) Applicant: Molly Sheehan, Port Colborne (CA)

(72) Inventor: Molly Sheehan, Port Colborne (CA)

(21) Appl. No.: 19/204,723

(22) Filed: May 12, 2025

Related U.S. Application Data
(60) Provisional application No. 63/647,246, filed on May 14, 2024.

(51) Int. Cl. A41B 9/00; A41B 9/04; A61F 13/496

(52) U.S. Cl. CPC A41B 9/008; A41B 9/04; A61F 13/496

(57) ABSTRACT

An underwear includes a first coverage portion, a second coverage portion, and a crotch panel extending between the first coverage portion and the second coverage portion and adapted to be arranged between of a wearer, covering a crotch area of the wearer. The underwear also includes a pair of tabs extending outwardly of the first coverage portion and configured to removably couple the first coverage portion and the second coverage portion.

### BACKGROUND

### 1. Technical Field

The present disclosure relates, generally, to an underwear, and more particularly relates to a restroom changeable menstrual underwear that can be easily removed in a public restroom.

### 2. Description of the Prior Art

Generally, horseback riding competitions require tan or white colored pants which make “leaks” highly visible. Riders, often, wear menstrual underwear to avoid leaks, however, once soiled, the underwear become uncomfortable, less effective and kind of gross. Competitions typically have washroom stalls or portable toilets that are too small and dirty to comfortably undress to change soiled garments.

### SUMMARY

In one aspect, an underwear is disclosed. The underwear includes a first coverage portion, a second coverage portion, and a crotch panel extending between the first coverage portion and the second coverage portion and adapted to be arranged between legs of a wearer, covering a crotch area of the wearer. The underwear further includes a pair of tabs extending outwardly of the first coverage portion and configured to removably couple the first coverage portion and the second coverage portion.

In some additional, alternative, or selectively cumulative embodiments, each of the pair of tabs includes an engagement layer adapted to engage with the second coverage portion to removably engage the first coverage portion and the second coverage portion.

In some additional, alternative, or selectively cumulative embodiments, the engagement layer is made of adhesive material.

In some additional, alternative, or selectively cumulative embodiments, the underwear further includes at least one flap attached to the second coverage portion and adapted to be arranged overlapping the pair of tabs and removably engaged with the pair of tabs. The pair of tabs are arranged sandwiched between the at least one flap and the second coverage portion in an engagement of the first coverage portion and the second coverage portion.

In some additional, alternative, or selectively cumulative embodiments, the at least one flap includes a pair of flaps arranged proximate to side edges of the second coverage portion.

In some additional, alternative, or selectively cumulative embodiments, the at least one flap includes a single flap attached to a top edge of the second coverage portion.

In some additional, alternative, or selectively cumulative embodiments, the at least one flap includes a coupling layer adapted to removably engage the at least one flap with the pair of tabs.

In some additional, alternative, or selectively cumulative embodiments, wherein the coupling layer is a made of adhesive material.

In some additional, alternative, or selectively cumulative embodiments, wherein each of the pair of tabs includes a retention layer adapted to facilitate the removable engagement of the associated tab with the at least one flap.

In some additional, alternative, or selectively cumulative embodiments, wherein the retention layer is a made of adhesive material.

In some additional, alternative, or selectively cumulative embodiments, the underwear further includes at least one attachment layer arranged attached to an outer surface of second coverage portion. The pair of tabs are removably engaged with the at least one attachment layer to removably engage the first coverage portion and the second coverage portion.

In some additional, alternative, or selectively cumulative embodiments, the at least one attachment layer is a made of adhesive material.

In some additional, alternative, or selectively cumulative embodiments, the at least one attachment layer includes a pair of attachment layers extending laterally inwardly towards each other from side edges of the second coverage portion, and the pair of tabs are removably engaged to the pair of attachment layers.

In some additional, alternative, or selectively cumulative embodiments, the at least one attachment layer includes a single attachment layer extending along a top edge of the second coverage portion between side edges of the second coverage portion, and the pair of tabs are removably engaged with the single attachment layer.

### BRIEF DESCRIPTION OF THE DRAWINGS

FIG. 1 illustrates a front perspective view of an underwear, in accordance with an embodiment of the disclosure;

FIG. 2 illustrates a top view of the underwear of FIG. 1 with a first coverage portion and a second coverage portion arranged disengaged from each other, in accordance with an embodiment of the disclosure;

FIG. 3 illustrates a side perspective view of an underwear, in accordance with an alternative embodiment of the disclosure; and

FIG. 4 illustrates a front perspective view of an underwear, in accordance with an alternative embodiment of the disclosure.

### DETAILED DESCRIPTION

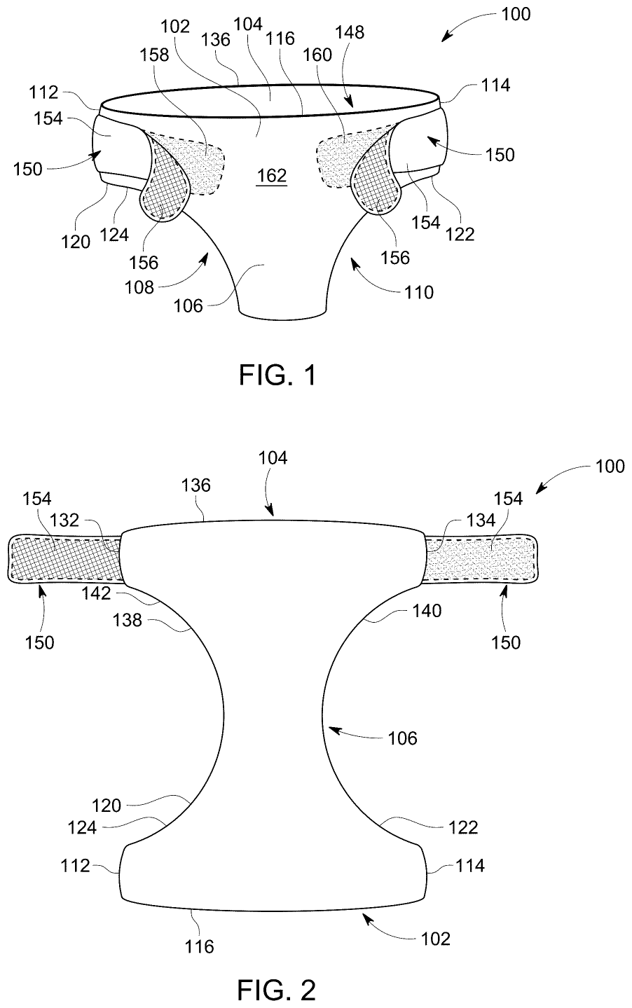

Referring to FIGS. 1 and 2, an underwear 100 is disclosed. The underwear 100 includes a front coverage portion 102, a rear coverage portion 104 and a crotch panel 106 connecting the front coverage portion 102 and the rear coverage portion 104. As used herein, the front coverage portion 102 refers to that part of the underwear which is generally located on the front of the wearer, and the rear coverage portion 104 refers to the portion of the underwear generally located at the rear of the wearer, covering hips of the user. The crotch panel 106 refers to that portion of the underwear which is generally located between the legs of the wearer. Further, the underwear defines a pair of leg openings 108, 110, shown in FIG. 1, through which the legs of the wear extends. The leg openings 108, 110 are defined between lower ends of the front and rear coverage portions 102, 104 and the crotch panel 106. In an embodiment, the crotch panel 106 is made of a leak proof material. The underwear 100 is made of a thinner material used to absorb menstrual discharge so that there is less bulk, distraction and discomfort for athletes while competing. Better leak protection and higher absorbency to guarantee a wearer stays leak free during the activity.

The front coverage portion 102, i.e., second coverage portion 102, includes a first side edge 112, a second side edge 114 disposed opposite to the first side edge 112, a top edge 116, and a bottom edge 120 having a first bottom edge portion 124 and a second bottom edge portion 122 extending on both sides of the crotch panel 106. Similarly, as shown in FIG. 2, the rear coverage portion 104 i.e., first coverage portion 104, includes a first side edge 132, a second side edge 134 disposed opposite to the first side edge 132, a top edge 136, and a bottom edge 138 having a first bottom edge portion 142 and a second bottom edge portion 140 extending on both sides of the crotch panel 106. It may be appreciated that front coverage portion 102 and the rear coverage portion 104 are removably coupled to each other at location proximate to the top edges 116, 136 such that the top edges 116, 136 of the two coverage portions 102, 104 are arranged facing each other defining a waist opening 148, shown in FIG. 1, of the underwear 100 when the two coverage portions 102, 104 are attached together. Similarly, the first bottom edge portions 124, 142 and the crotch panel 106 together defines a first leg opening 108 of the underwear 100, while the second bottom edge portions 122, 140 and the crotch panel 106 defines a second leg opening 110 of the underwear 100 when the two coverage portions 102, 104 are attached together.

To facilitate the removable coupling of the front coverage portion 102 and the rear coverage portion 104, the underwear 100 includes a pair of tabs, for example, one of the tabs 150 extending outwardly from the first side edge 132 of the rear coverage portion 104, and another of the tabs 150 extending outwardly from the second side edge 134 of the rear coverage portion 104. In the illustrated embodiment, the tabs 150 are arranged proximate to the top edge 136 of the rear coverage portion 104, and each includes a first layer 154 and a second layer 156, also referred to as engagement layer 156. The first layer 154 is an outer layer made of fabric, such as, but not limited to, cotton, spandex, etc., while the second layer 156 i.e., the engagement layer 156, is an inner layer made of an adhesive material, for example, silicon, Velcro, etc., to facilitate a removable engagement of the tabs 150 with front coverage portion 102. It may be appreciated that the tabs 150 of the underwear 100 act as side adjuster of a pant or trouser to enables the proper fit of the underwear around the waist of the wearer. The underwear 100 can be removed without completely removing the pants. For removing the underwear 100, the person opens the top portion of the pant, and then disengages the tabs 150 from the front coverage portion 102 by pulling the tabs 150 away from the front coverage portion 102, thereby disengaging the tabs 150 and hence the rear coverage portion 104 from the front coverage portion 102. Thereafter, the underwear 100 is removed by pulling the open underwear.

In the illustrated embodiment, the underwear 100 includes a pair of attachment layers 158, 160, shown in FIG. 1, arranged on an outer surface 162 of the front coverage portion 102 and extending towards each other from the first and second side edges 112, 114 of the front coverage portion 102. The location and dimensions of the attachment layers 158, 160 correspond to the location and dimensions of the tabs 150. In the embodiment, the attachment layers 158, 160 facilitates secure engagement of the tabs 150 with the front coverage portion 102. In some embodiments, the attachment layers 158, 160 are made of an adhesive material, for example, silicon, Velcro, etc.,

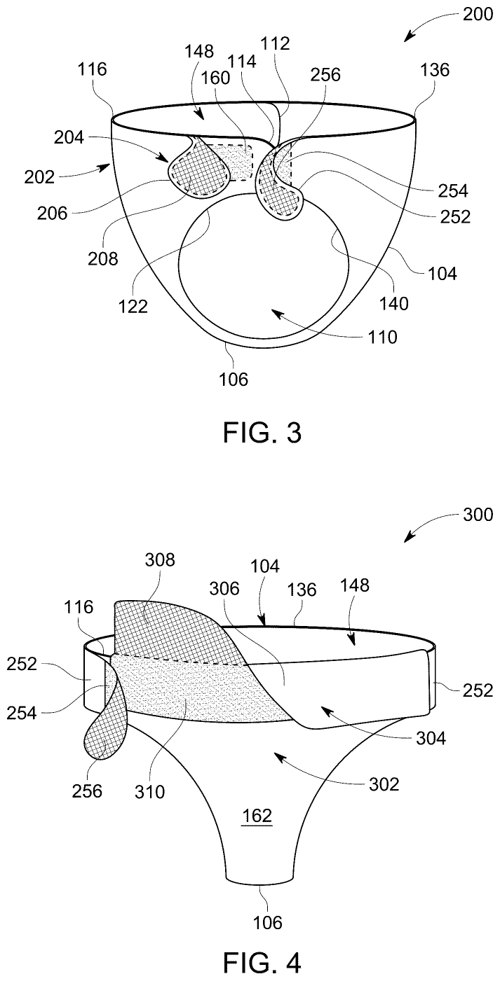

Referring to FIG. 3, an underwear 200 according to another embodiment is shown. The underwear 200 is similar to the underwear 100 shown in FIGS. 1 and 2, except that tabs 252 of the underwear 200 are different from the tabs 150 of the underwear 100 in that each of the tabs 252 includes a first layer 254, i.e., a retention layer 254, and a second layer 256 i.e., engagement layer 256, each made of an adhesive material, for example, silicon, Velcro, etc. Further, the underwear 200 includes a pair of flaps 204 arranged proximate to the first side edge 112 and the second side edge 114 of a front coverage portion 202 and arranged overlapping side portions of the front coverage portion 202. In the embodiment, a first end of the flaps 204 is arranged inwardly of the associated first and second side edges 112, 114, and the flaps 204 extend toward the associated first and second side edges 112, 114 from the first end such that second ends of the flaps 204 is arranged disengaged from the front coverage portion 202. In the embodiment, each of the flaps 204 includes a first layer 206 i.e., outer layer, made of fabric, and a second layer 208 i.e., coupling layer 208, made of adhesive material, for example, silicon, Velcro, etc. To connect the front coverage portion 202 and the rear coverage portion 104, the second layer 256 i.e., engagement layer 256, of each tab 252 is attached to the associated one of attachment layers 158, 160 of the front coverage portion 202, and the first layer 254 i.e., retention layer 254, of each tab 252 is attached to the second layer 208 i.e., coupling layer 208 of the associated flap 204. Accordingly, in the engagement of the front coverage portion 202 and the rear coverage portion 104, each of the tabs 252 are arranged sandwiched between the outer surface 162 of the front coverage portion 202 and the associated flap 204. The sandwiched engagement enables a secure engagement of the front coverage portion 202 with the rear coverage portion 104.

Referring to FIG. 4, an underwear 300 according to another embodiment is shown. The underwear 300 is similar to the underwear 200 except that the pair of flaps 204 are omitted from the underwear 200. Instead, the underwear 300 includes a single flap 304 extending along the top edge 116 of the front coverage portion 202 and adapted to be folded downwardly to the to the top edge 116. As shown, the flap 304 includes a first layer 306 i.e., outer layer made of fabric and a second layer 308, i.e., coupling layer 308, made of adhesive material, for example, silicon, Velcro, etc. Also, the underwear 300 includes a single attachment layer 310 extending along the top edge 116 of a front coverage portion 302 and attached to the front coverage portion 302. The attachment layer 310 is made of adhesive material and is adapted to engage with the second layers 256 i.e., engagement layers 256, of the tabs 252. Also, the second layer 308 i.e., coupling layer 308 of the flap 304 is adapted to engage with the first layers 254, i.e., retention layers 254, of the tabs 252. Accordingly, the tabs 252 are arranged sandwiched between the attachment layer 310 of the front coverage portion 302 and the flap 304. Although the flap 304 is shown to be arranged overlapping an outer surface of the front coverage portion 302, it may be envisioned that the flap 304 may be arranged inwardly of the underwear 300 i.e., overlapping an inner surface of the front coverage portion 302, and in such a case, the attachment layer 310 is attached to an inner surface of the front coverage portion 302.

Although, the tabs 150, 252 are shown to be attached to the front coverage portion 102, 202, 302 of the underwear 100, 200, 300, it may be appreciated that the tabs 150, 252 may be attached to the rear coverage portion 104 of the underwear 100, 200, 300 and accordingly, the attachment layer 310, 158, 160, the flap 304, 204 are attached to the front coverage portion 102, 202, 302. Also, in some embodiments, one or more of the attachment layer 158, 160, 310, the engagement layer 156, 256, the retention layer 254, the coupling layer 208, 308 may be omitted while allowing the removable engagement of the tabs with the front coverage portion 102. For example, for the embodiment, shown in FIG. 1, either the attachment layers 158, 160 or the engagement layers 156 of the tabs 150 may be omitted, while still allowing the engagement of the tabs 150 with the front coverage portion 102. Similarly, in the embodiment shown in FIG. 3, either the retention layers 254 of the tabs 252 or the coupling layers 208 of the flaps 204 may be omitted. Furthermore, in some embodiments, shown in FIG. 4, either the engagement layers 256 of the tabs 252 or the attachment layer 310 may be omitted. Similarly, either the retention layers 254 of the tabs 252 or the coupling layer 308 of the flap 304 may be omitted.

It should be understood that the foregoing description is only illustrative of the aspects of the disclosed embodiments. Various alternatives and modifications can be devised by those skilled in the art without departing from the aspects of the disclosed embodiments.

What is claimed is:

1. An underwear, comprising:
a first coverage portion;
a second coverage portion;
a crotch panel extending between the first coverage portion and the second coverage portion and adapted to be arranged between legs of a wearer, covering a crotch area of the wearer; and
a pair of tabs extending outwardly of the first coverage portion and configured to removably couple the first coverage portion and the second coverage portion.

2. The underwear of claim 1, wherein each of the pair of tabs includes an engagement layer adapted to engage with the second coverage portion to removably engage the first coverage portion and the second coverage portion.

3. The underwear of claim 2, wherein the engagement layer is made of adhesive material.

4. The underwear of claim 1 further comprising at least one flap attached to the second coverage portion and adapted to be arranged overlapping the pair of tabs and removably engaged with the pair of tabs, wherein the pair of tabs are arranged sandwiched between the at least one flap and the second coverage portion in an engagement of the first coverage portion and the second coverage portion.

5. The underwear of claim 4, wherein the at least one flap includes a pair of flaps arranged proximate to side edges of the second coverage portion.

6. The underwear of 4, wherein the at least one flap includes a single flap attached to a top edge of the second coverage portion.

7. The underwear of claim 4, wherein the at least one flap includes a coupling layer adapted to removably engage the at least one flap with the pair of tabs.

8. The underwear of claim 7, wherein the coupling layer is a made of adhesive material.

9. The underwear of claim 4, wherein each of the pair of tabs includes a retention layer adapted to facilitate the removable engagement of the associated tab with the at least one flap.

10. The underwear of claim 9, wherein the retention layer is a made of adhesive material.

11. The underwear of claim 1 further includes at least one attachment layer arranged attached to an outer surface of second coverage portion, wherein the pair of tabs are removably engaged with the at least one attachment layer to removably engage the first coverage portion and the second coverage portion.

12. The underwear of claim 11, wherein the at least one attachment layer is a made of adhesive material.

13. The underwear of claim 11, wherein the at least one attachment layer includes a pair of attachment layers extending laterally inwardly towards each other from side edges of the second coverage portion, and the pair of tabs are removably engaged to the pair of attachment layers.

14. The underwear of claim 11, wherein the at least one attachment layer includes a single attachment layer extending along a top edge of the second coverage portion between side edges of the second coverage portion, and the pair of tabs are removably engaged with the single attachment layer.
